(19) United States
(12) Patent Application Publication
(10) Pub. No.: US 2023/0084426 A1
(43) Pub. Date: Mar. 16, 2023

(54) APPARATUS FOR THE CONTROL OF A TRAINING DEVICE

(71) Applicant: BitifEye Digital Test Solutions GmbH, Böblingen (DE)

(72) Inventors: Valentina Unakafova, Stuttgart (DE); Anton Unakafov, Stuttgart (DE); Alexander Schmitt, Sindelfingen (DE); Thomas Marik, Herrenberg (DE); Balthasar Stelzner, Böblingen (DE); Joachim Köninger, Horb (DE); Max Batt, Tübingen (DE); Julien Henaut, Stuttgart (DE)

(21) Appl. No.: 17/983,374

(22) Filed: Nov. 8, 2022

Related U.S. Application Data
(63) Continuation of application No. PCT/EP2022/053322, filed on Feb. 11, 2022.

(30) Foreign Application Priority Data
Feb. 25, 2021 (DE) 10 2021 104 520.7

(51) Int. Cl. A63B 24/00; A63B 22/06; A63B 22/00; A63B 23/04

(52) U.S. Cl. CPC A63B 24/0087; A63B 22/0605; A63B 22/0076; A63B 23/0476; A63B 2024/0093; A63B 2220/18; A63B 2220/34; A63B 2230/045

(57) ABSTRACT

An apparatus for controlling of a training device including a training device configured to absorb a mechanical power applied by a person undertaking physical training, an assistance unit configured to assist the training and/or to make the training more difficult, and an exertion measuring apparatus configured to measure mechanical exertion data of an effort applied by the person during the training, a body sensor configured to measure physiological data of the body of the person, a computing unit configured, with an optimization algorithm, to adjust coefficients, a summand, and delays to prepare a prediction of the physiological data based on a model, and a control unit configured to take a predetermined reference variable for the physiological data, to take the prediction as a control variable, and to control an assistance of the assistance unit as a manipulated variable.

### CROSS REFERENCE TO RELATED APPLICATIONS

This application is a continuation application of international patent application PCT/EP2022/053322, filed Feb. 11, 2022, designating the United States and claiming priority to German application 10 2021 104 520.7, filed Feb. 25, 2021, and the entire content of both applications is incorporated herein by reference.

### TECHNICAL FIELD

The disclosure relates to an apparatus for controlling a training device.

### BACKGROUND

A large number of training devices exist with which a person can train and thereby improve his fitness. An electric bicycle may be mentioned as an example. Other examples include a bicycle ergometer, an abductor/adductor machine, and an arm strength traction device. During a training session it is crucial that the person makes enough effort, so that the training truly does lead to an improvement in fitness, but also that excessive stress that can lead to physical harm to the person is avoided. It must be remembered that an optimum stress range can differ greatly from one person to another. It is important that the training device is used correctly during training or that it is properly adjusted, so that the person makes sufficient effort but at the same time is not overstressed. In the ideal case, the training device should be designed such that it can be adjusted both for a person suffering from a weak heart as well as for a person who is working in high-performance sport. One example of an incorrect usage of the training device would be when the power of the motor of the electric bicycle is set too high. As a result, the person does not make enough effort, but at the same time rides at a relatively high speed that is associated with an increased risk of accident.

### SUMMARY

An object of the disclosure therefore is to provide an apparatus with a training device that is controlled in such a way that a person training with the training device can make sufficient effort but, at the same time, excessive stress of the person can be avoided.

An apparatus according to the disclosure for controlling a training device includes:
the training device that is configured to absorb a mechanical power applied by a person undertaking physical training, wherein the training device includes an assistance unit that is configured to assist the training and/or to make the training more difficult, wherein the training device include an exertion measuring apparatus that is configured to measure mechanical exertion data BD(t) of an effort applied by the person during the training, wherein t is the time,
a body sensor that is configured to measure physiological data PD(t) of the body of the person,
a computing unit in which a mathematical model in the form mPD(t+T) is stored, wherein the computing unit is configured, with an optimization algorithm, to adjust mPD(t+T) and the delay T individually for each person in such a way that mPD(t+T) approaches the measured physiological data PD(t+T), and to prepare a prediction mPD(t+T) of the physiological data PD(t+T) on the basis of the model, and
a control unit that is configured to take a predetermined reference variable for the physiological data PD(t), to take the prediction mPD(t+T) as a control variable, and to control an assistance u(t) of the assistance unit as a manipulated variable.

It is typical that the equations:

$$m PD ( t + T ) = {a}_{10} + {\sum }_{x} {B}_{x} ( t ) \\ and {B}_{1} ( t ) = {\sum }_{i = 1}^{j} {a}_{1 i} * ( {\sum }_{d = 0}^{{D}_{i}} BD ( t - {τ}_{1 i} - d * {K}_{i} ) / ( {D}_{i} + 1 ) ) \\ and {B}_{2} ( t ) = {\sum }_{i = 1}^{k} {a}_{2 i} * PD ( t - {τ}_{2 i} )$$

apply, wherein the computing unit is configured, with the optimization algorithm, to adjust the coefficients aₓᵢ, the summand a₁₀ and the delays τₓᵢ at least partially for each person individually in such a way that mPD(t+T) approaches the measured physiological data PD(t+T). An averaging of Dᵢ+1 measuring points, which have a time interval is performed in the term B₁(t). The values for Dᵢ can, for example, be chosen from a range from 0 up to 60. The values for the time interval Kᵢ can, for example, be chosen from a range from 0.2 seconds up to 2 seconds.

Because the apparatus takes the prediction mPD(t+T) in which the time t+T lies by the delay time Tin the future as the control variable, the control unit can react much more quickly to changes in the training than would be the case if the physiological data PD(t) were taken as the control variable. As a result, control deviations of the control variable from the reference variable can be kept much lower than would be the case if the physiological data PD(t) were to be taken as the control variable. Because the control unit is configured to adjust the coefficients aₓᵢ, the summand a₁₀, the delays τₓᵢ and the delay T individually for each person, the control deviations can be kept low for every one of the persons. Different persons react with different speeds to a change of a loading acting on the person from outside generated, for example, by the training device. If the person is relatively untrained, the person will tend to react slowly to the change, while if the person is relatively trained, the person will react to the change relatively quickly. Because the computing unit is not only configured to adjust the coefficients aₓᵢ and the summand a₁₀ individually for each person, but also the delays τₓᵢ and the delay T, the model can reflect the fact that different persons react at different speeds to the changes in the loading. As a result, the prediction has a particularly high accuracy for each person, whereby control deviations are also particularly low. The only thing that is still necessary is to specify the appropriate reference variable for the physiological PD(t) for each person, while it is conceivable that the reference variable does change over time. A sports physician or a physiotherapist can, for example, be employed for setting the reference variable. Because the control deviations are particularly low, it is now possible for the training device to be controlled in such a way that the person makes sufficient effort, so that fitness of the person is improved, and that excessive stress on the person is avoided.

The assistance u(t) can be positive, whereby the training is assisted, and/or negative, whereby the training is made more difficult. An electric motor of an electric bicycle is an example of an assistance unit that is configured to assist the training. In this case, the assistance could, for example, be power applied by the electric motor. A brake of a bicycle ergometer is an example of an assistance unit that is configured to make the training more difficult. In this case, the assistance can, for example, be a braking power. An example of an assistance unit that is configured to support the training and to make it more difficult is an electric motor of an electric bicycle that is configured to perform recovery, i.e., to convert a pedalling power of the person into electrical current. In order to keep the control deviation particularly low, it is typical that the assistance unit is configured to control the assistance u(t) in small increments. The increments can, for example, be a maximum of 3%, in particular a maximum of 1.5% or a maximum of 1%. 100% here represents a maximum assistance u(t) in the case in which the assistance unit is configured to assist the training. In the case in which the assistance unit is configured to make the training more difficult, −100% corresponds to a maximum opposition to the training.

The exertion data BD(t) characterize a mechanical effort applied by the person during the training to overcome the loading. The exertion data BD(t) are zero when the person is resting. The physiological data includes variables that characterize the way in which systems and/or subsystems in the body of the person are functioning, and that can be measured with a sensor. The system or the subsystem can be the cardiorespiratory system or a part thereof, or can be the musculoskeletal system or a part thereof. The physiological data PD(t) can, example, be a heart rate. There are several variables, such as a knee adduction torque and/or a knee abduction torque, that may come into question both for the exertion data BD(t) as well as for the physiological data PD(t).

It is typical if j is chosen from the range between 2 and 5. It has been found that only low computing power is needed for j=2, although a sufficient precision is nevertheless achieved for the prediction, whereas for j=5 a greater precision is achieved for the prediction.

It is typical if k is chosen from the range between 1 and 4. It has been found that only low computing power is needed for k=1, although a sufficient precision is nevertheless achieved for the prediction, whereas for k=4 a greater precision is achieved for the prediction.

The training device typically includes an altimeter that is configured to measure the altitude h(t) of the training device, wherein B₃(t)=Σᵢ₌₁^{l}a ₃ᵢ*h(t−τ₃ᵢ) applies in the model. Because the altitude h(t) has an effect on the loading, the control deviations can be further reduced through the use of B₃(t). The provision of the altimeter is particularly relevant when the training device is the electric bicycle. The altimeter can, for example, be realized by a GPS receiver. The GPS receiver can, for example, be part of a smartphone. It is particularly typical if l is chosen from the range between 1 and 4. It has been found that only low computing power is needed for l=1, although a sufficient precision is nevertheless achieved for the prediction, whereas for l=5 a greater precision is achieved for the prediction.

The training device typically includes a temperature sensor for measuring the temperature Temp(t) in the surroundings of the training device, wherein

$${B}_{4} ( t ) = {\sum }_{i = 1}^{m} {a}_{4 i} * T e mp ( t - {τ}_{4 i} )$$

in the model. Because the temperature Temp(t) has a large effect on the loading, the control deviations can be further reduced through the use of B₄(t). The provision of the temperature sensor is particularly relevant when the training device is provided for use in the open such as in the case, for example, of the electric bicycle. It is particularly typical if m is chosen from the range between 1 and 2. It has been found that only low computing power is needed for m=1, although a sufficient precision is nevertheless achieved for the prediction, whereas for m=2 a greater precision is achieved for the prediction.

It is typical for the training device to include an inclinometer that is configured to measure an incline N(t) of the training device, and

$${B}_{5} ( f ) = {\sum }_{i = 1}^{n} {a}_{5 i} * N ( t - {τ}_{5 i} )$$

in the model. The inclinometer can, for example, include an incline calculation unit that is configured to determine the incline N(t) from the time derivative of the altitude dh(t)/dt. It is alternatively conceivable that the inclinometer is part of a smartphone. It is also conceivable that the inclinometer is permanently installed in the training device. It is particularly typical if n is chosen from the range between 1 and 4. It has been found that only low computing power is needed for n=1, although a sufficient precision is nevertheless achieved for the prediction, whereas for n=4 a greater precision is achieved for the prediction.

It is typical that the delays τₓ₁ are zero, and that all the delays τₓᵢ are adjusted for i>1. It is typical that the computing unit is configured to prepare the prediction mPD(t+T) for the time t+T that lies at least T=5 s in the future.

It is typical for the computing unit to be configured to adjust, on the basis of the optimization algorithm, the coefficients aₓᵢ, the summand a₁₀, the delays τₓᵢ and the delay T after the training session, making use of the exertion data BD(t) ascertained in a plurality of training sessions and the physiological data PD(t) ascertained in the plurality of training sessions, as well as, optionally, of the altitude h(t) ascertained in the plurality of training sessions, the temperature Temp(t) ascertained in the plurality of training sessions and/or the incline N(t) ascertained in the plurality of training sessions, in order to take an underlying fitness of the person into consideration. The plurality of training sessions can, for example, be all the training sessions carried out by the person. It is, alternatively, conceivable that the plurality of training sessions is a number of the training sessions most recently carried out.

It is typical that the computing unit is configured to adjust the coefficients aₓᵢ, the summand a₁₀, the delays τₓᵢ and the delay T after the training session with the optimization algorithm 11, which has the steps of: a) specifying in each case a plurality of discrete values for each of the coefficients aₓᵢ, for each of the delays τₓᵢ, for the summand a₁₀ and for the delay T; b) setting aₓᵢ, a₁₀, τₓᵢ and T to one of the values; c) calculating mPD(t+T) on the basis of the model; d) calculating a modelling error between the measured physiological data PD(t+T) and mPD(t+T) for a plurality of t; e) repeating steps b) to d) for all combinations of the values; f) choosing those values for aₓᵢ, a₁₀, τₓᵢ and T, that result in the lowest modelling error. While it is true that this is a computing-intensive method, the values aₓᵢ, a₁₀, τₓᵢ and T can nevertheless be determined with a high accuracy, so that the control deviations are particularly small. It is particularly typical that underestimation errors are weighted more strongly than overestimation errors in step d).

The computing unit is typically configured to adjust, with an algorithm for adjusting a current fitness, the coefficients aₓᵢ and the summand a₁₀ during a training session, making use of the exertion data BD(t) ascertained in the training session and the physiological data PD(t) ascertained in the training session, as well as, optionally, of the altitude h(t) ascertained in the training session, the temperature Temp(t) ascertained in the training session and/or the incline N(t) ascertained in the training session, in order to take the current fitness of the person into consideration. The control deviations can be kept particularly low by taking the current fitness into consideration.

It is particularly typical that the computing unit is configured to determine, with the algorithm for adjusting the current fitness, a difference Diff(t)=mPD(t)−PD(t) between the prediction of the physiological data mPD(t) and the measured physiological data PD(t), and if the difference Diff(t) exceeds a threshold value Threshold₁>0, to correct the coefficients aₓᵢ by adding a respective constant const₁ₓᵢ, as well as to correct the summand a₁₀ by adding a constant const₁₀ and, if the difference Diff(t) falls below a threshold value of Threshold_{M}<0 to correct the coefficients aₓᵢ by adding a respective constant cons_{Mxi,} as well as to correct the summand a₁₀ by adding a constant const_{M0}. This, advantageously, is not a very computing-intensive method, and is also suitable for being carried out during the training session. More threshold values can also be provided. Appropriate program code can, for example, look like this:

```
if (Diff(t)>Threshold₁)
  aₓ₁ = aₓ₁ + const₁ₓ₁
  aₓ₂ = aₓ₂ + const₁ₓ₂,
  ...
elseif (Diff(t)>Threshold₂)
  aₓ₁ = aₓ₁ + const₂ₓ₁,
  aₓ₂ = aₓ₂ + const₂ₓ₂,
  ...
...
elseif (Diff(t)>Threshold_{M-1})
  aₓ₁ = aₓ₁ + const_{(M-1)x1},
  aₓ₂ = aₓ₂ + const_{(M-1)x2},
  ...
elseif (Diff(t)<Threshold_{M})
  aₓ₁ = aₓ₁ + const_{Mx1},
  aₓ₂ = aₓ₂ + const_{Mx2},
  ...
...
elseif (Diff(t)<Threshold_{M+K})
  aₓ₁ = aₓ₁ + const_{(M+K)x1},
  aₓ₂ = aₓ₂ + const_{(M+K)x2},
  ...
End
```

With each if-query here, all of the coefficients aₓᵢ and the summand a₁₀ are corrected, and the following applies:

Threshold₁>Threshold₂> . . . >Threshold_{M-1}>Threshold_{M+K}> . . . >Threshold_{M+1}>Threshold_{M}

The control unit is, typically, a PID controller. The PID controller is particularly suitable for controlling the physiological data PD(t), since its integral term contributes to gradually reducing the control deviation, while its differential term makes it possible to overcome control deviations even before they actually occur. It is particularly typical here for the PID controller to be configured to determine the assistance u(t) according to

$$u ( t ) = {K}_{P} * {f}_{1} ( e ( t ) ) + {K}_{I} * {\int }_{τ = 0}^{τ = t} {f}_{2} ( e ( τ ) ) d τ + {K}_{D} * \frac{d {f}_{3} ( e ( t ) )}{dt}$$

wherein K_{P}, K_{I} and K_{D} are control parameters, wherein e(t) is the control deviation at time t, wherein the functions f₁(e), f₂(e) and f₃(e) are selected such that underestimation errors are weighted more strongly than overestimation errors. As a result, deviations of the control variable to values larger than the reference variable are less probable than deviations of the control variable that have lower values than the reference variable. The excessive stress that could cause physical injury to the person can thereby be avoided. It is particularly typical that

$${f}_{1} ( e ) = {\sum }_{i = 0}^{p} {c}_{i 1} * {e}^{i} \\ and \\ {f}_{2} ( e ) = {\sum }_{i = 0}^{q} {c}_{i 2} * {e}^{i}$$

and f₃(e)=0 for e<0 and f₃(e)=e for e≥0, while in f₁(e) and f₂(e) the polynomial can be different in different ranges of e.

It is particularly typical that the computing unit is configured to adjust the control parameters K_{P}, K_{I} and K_{D} individually for each person. In this way it can be achieved that the control deviations are particularly low for each person.

The computing unit is typically configured to carry out a calibration method in which a step response of the physiological data PD(t) is generated by an abrupt change in the manipulated variable, wherein the computing unit is typically configured to determine the control parameters K_{P}, K_{I} and K_{D} from the step response. The computing unit can be configured to record the physiological data PD(t) continuously to generate the step response. The computing unit is configured to switch the assistance unit from a constant first assistance u₁ to a constant second assistance u₂ at a time T₀, in order thereby to bring about the abrupt change in the manipulated variable. For example, u₁ can be from 80% to 100% and u₂ can be from 0% to 20%. The person can be shown information here indicating that they should train as far as possible at a constant frequency, for example a pedalling frequency. The computing unit is configured to wait long enough, both during the first assistance u₁ and during the second assistance u₂, for the physiological data PD(t) to have stabilized around a value of PD₁ before the changeover, and around a value of PD₂ after the changeover. The computing unit can be configured to wait at least 2 minutes both before and after the changeover. It is moreover conceivable for the computing unit to be configured to generate a second step response. For this purpose, the computing unit can be configured so that after the exertion data BD(t) or the physiological data PD(t) have stabilized following the abrupt change in the manipulated variable, it switches the assistance over from u₂ to u₁ and waits again until the physiological data PD(t) have stabilized.

It is typical that the computing unit is configured to identify at least one abrupt change in the manipulated variable and the resulting step response of the physiological data PD(t) after a training session, wherein the computing unit is configured to determine the control parameters K_{P}, K_{I} and K_{D} from the at least one step response. It is conceivable that the computing unit is configured to use the calibration method for a coarse adjustment of the control parameters K_{P}, K_{I} and K_{D} and to use the at least one step response identified outside the calibration method following the training session in order to perform a fine adjustment of the control parameters K_{P}, K_{I} and K_{D}.

Typically the exertion data BD(t) include a power, in particular a pedalling power in the case of a bicycle, in particular of an electric bicycle, or in the case of a bicycle ergometer, a running power, a rowing power, a speed, a torque, a rotation speed, an angular speed and/or a knee abduction torque.

It is typical that the assistance unit includes an electric motor, a gearbox and/or a brake.

It is typical that the physiological data PD(t) include a heart rate, a heart rate variability, an electrocardiogram, an oxygen saturation of the blood, a blood pressure, a neurological activity, in particular an electroencephalography, a knee abduction torque, an adduction, in particular a knee adduction, and/or a knee bend.

### BRIEF DESCRIPTION OF THE DRAWINGS

The disclosure will now be described with reference to the drawings wherein:

FIG. 1 shows an overview of an apparatus according to an exemplary embodiment of the disclosure.

FIG. 2 shows a detail of the overview according to an exemplary embodiment of the disclosure.

FIG. 3 shows a plot of f₁(e) and f₂(e).

FIG. 4 shows a plot of f₃(e).

FIG. 5 shows a plot of a step response of the physiological data PD(t) that is generated by an abrupt change in the manipulated variable.

FIG. 6 shows a plot of various measured variables recorded during a training session.

### DESCRIPTION OF EXEMPLARY EMBODIMENTS

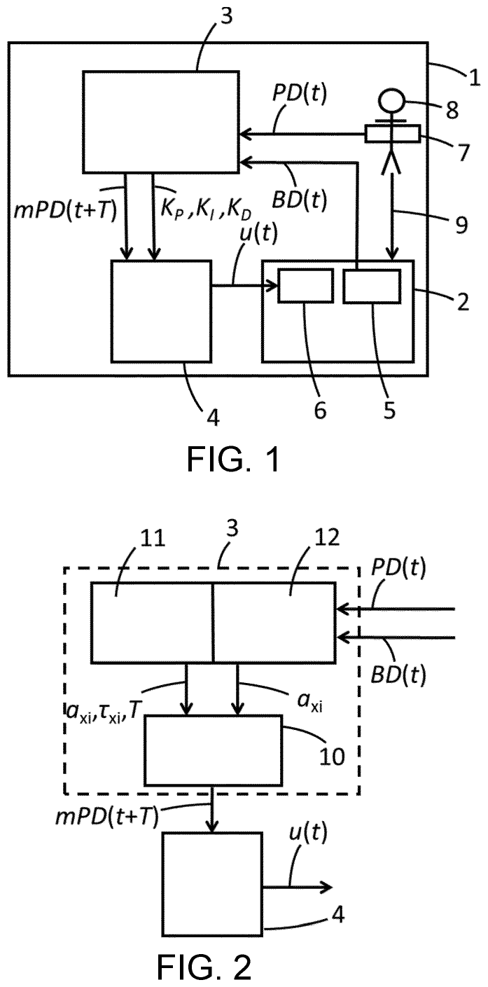

FIGS. 1 and 2 show that the apparatus 1 for controlling a training device 2 includes:
the training device 2 that is configured to absorb a mechanical power 9 applied by a person 8 undertaking physical training, wherein the training device 2 includes an assistance unit 6 that is configured to assist the training and/or to make the training more difficult, wherein the training device 2 includes an exertion measuring apparatus 5 that is configured to measure mechanical exertion data BD(t) of an effort applied by the person during the training, wherein t is the time,
a body sensor 7 that is configured to measure physiological data PD(t) of the body of the person 8,
a computing unit 3 in which a mathematical model of the form

$$m PD ( t + T ) = {a}_{10} + {\sum }_{x} {B}_{x} ( t )$$

is stored, in which

$${B}_{1} ( t ) = {\sum }_{i = 1}^{j} {a}_{1 i} * ( {\sum }_{d = 0}^{{D}_{i}} BD ( t - {τ}_{1 i} - d * {K}_{i} ) / ( {D}_{i} + 1 ) ) \\ and {B}_{2} ( t ) = {\sum }_{i = 1}^{k} {a}_{2 i} * PD ( t - {τ}_{2 i} )$$

wherein the computing unit 3 is configured, with an optimization algorithm 11, to adjust the coefficients aₓᵢ, the summand a₁₀, the delays τₓᵢ at least partially, and the delay T individually for each person in such a way that mPD(t+T) approaches the measured physiological data PD(t+T), and to prepare a prediction mPD(t+T) of the physiological data PD(t+T) on the basis of the model, and
a control unit 4 that is configured to take a predetermined reference variable for the physiological data PD(t), to take the prediction mPD(t+T) as a control variable, and to control an assistance u(t) of the assistance unit 6 as a manipulated variable. An averaging of Dᵢ+1 measuring points, which have a time separation Kᵢ, is performed in the term B₁(t).

The training device 2 can include an altimeter that is configured to measure the altitude h(t) of the training device 2, and can be

$${B}_{3} ( t ) = {\sum }_{i = 1}^{l} {a}_{3 i} * h ( t - {τ}_{3 i} )$$

in the model. Additionally, the training device 2 can include a temperature sensor that is configured to measure the temperature Temp(t) in the surroundings of the training device 2, and can be

$${B}_{4} ( t ) = {\sum }_{i = 1}^{m} {a}_{4 i} * Temp ( t - {τ}_{4 i} )$$

in the model. The training device 2 can include an inclinometer that is configured to measure an incline N(t) of the training device 2, and can be

$${B}_{5} ( t ) = {\sum }_{i = 1}^{n} {a}_{5 i} * N ( t - {τ}_{5 i} )$$

in the model.

The control unit can, for example, be a PID controller. The PID controller can, for example, be configured to determine the assistance u(t) in accordance with

$$u ( t ) = {K}_{P} * {f}_{1} ( e ( t ) ) + {K}_{I} * {\int }_{τ = 0}^{τ = t} {f}_{2} ( e ( τ ) ) d τ + {K}_{D} * \frac{d {f}_{3} ( e ( t ) )}{dt}$$

wherein K_{P}, K_{I} and K_{D} are control parameters, wherein e(t) is the control deviation at time t, wherein the functions f₁(e), f₂(e) and f₃(e) are selected such that underestimation errors are weighted more strongly than overestimation errors. Here it is possible that

$${f}_{1} ( e ) = {\sum }_{i = 0}^{p} {c}_{i 1} * {e}^{i} \\ and \\ {f}_{2} ( e ) = {\sum }_{i = 0}^{q} {c}_{i 2} * {e}^{i}$$

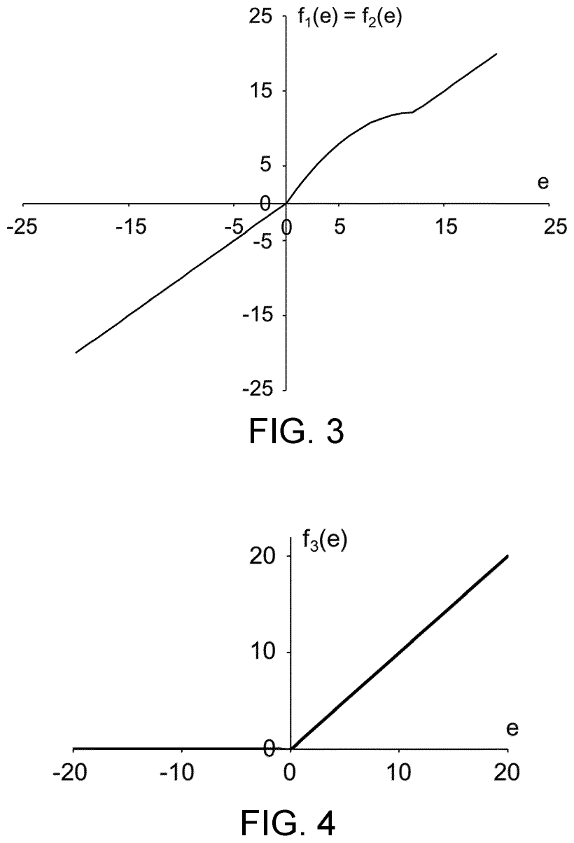

and f₃(e)=0 for e<0 and f₃(e)=e for e≥0, while in f₁(e) and f₂(e) the polynomial can be different in different ranges of e. FIG. 3 shows an exemplary plot of f₁(e)=f₂(e), and FIG. 4 shows an exemplary plot of f₃(e). As can be seen from FIG. 3, the functions f₁(e) and f₂(e) can have a bisector and only lie above the bisector in a range 0<e<E₁ or 0<e<E₂ respectively. Particularly when the physiological data are a heart rate, the following can, for example, apply: f₁(e)=f₂(e)=e for e>12 or e<0 and f₁(e)=f₂(e)=2*e−0.082*e² for 0≤e≤12. As can be seen from FIG. 4, it is, for example, possible for f₃(e) to be governed by f₃(e)=e for e>0 and f₃(e)=0 for e≤0.

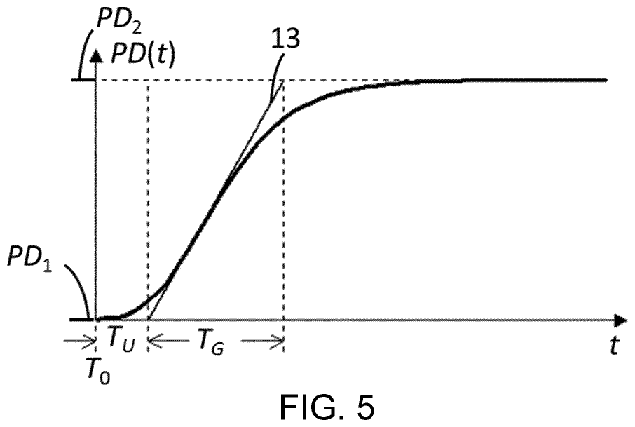

It is conceivable that the computing unit 3 is configured to adjust the control parameters K_{P}, K_{I} and K_{D} individually for each person 8. For this purpose, the computing unit 3 can be configured to carry out a calibration method in which a step response of the physiological data PD(t) is generated by an abrupt change in the manipulated variable at a time T₀, wherein the computing unit 3 is configured to determine the control parameters K_{P}, K_{I} and K_{D} from the step response. An exemplary step response is illustrated in FIG. 5. The computing unit 3 can be configured to record the physiological data PD(t) continuously to generate the step response. The computing unit 3 can be configured to switch the assistance unit 6 from a constant first assistance u₁ to a constant second assistance u₂, in order thereby to bring about the abrupt change in the manipulated variable. For example, u₁ can be from 80% to 100% and u₂ can be from 0% to 20%. The person can be shown information here indicating that they should train as far as possible at a constant frequency, for example a pedalling frequency. The computing unit 3 can be configured to wait long enough, both during the first assistance u₁ and during the second assistance u₂, for the physiological data PD(t) to have stabilized around a value of PD₁ before the changeover and around a value of PD₂ after the changeover. The computing unit 3 can be configured to wait at least 2 minutes both before and after the changeover. To determine the control parameters from the step response, the computing unit 3 can be configured to apply an inflection tangent 13 to the step response. Before applying the inflection tangent 13, PD(t) can be adjusted by a function, for example a polynomial, and the inflection tangent 13 can be applied to the adjusted function. A method of least square errors can be employed to adjust the function. The point of intersection of the inflection tangent 13 with PD(t)=PD₁ determines a delay duration T_{U} that starts at T₀, and the point of intersection of the inflection tangent 13 with PD(t)=PD₂ determines a settling duration T_{G} that starts at the end of T_{U}. The control parameters can now be determined, for example according to K_{P}=1.2*T_{G}/(K_{S}*T_{U}), K₁=0.6*T_{G}(K_{S}*(T_{U})²) and K_{D}=0.6*T_{G}/K_{S}, wherein K_{S} is the amplification factor and can be calculated as the ratio of the control parameter change to the assistance change.

It is conceivable that the computing unit is configured to identify at least one abrupt change in the manipulated variable and the resulting step response of the physiological data PD(t) or of the exertion data BD(t) after a training session, wherein the computing unit is configured to determine the control parameters K_{P}, K_{I} and K_{D} from the at least one step response. It is also conceivable that the computing unit is configured to use the calibration method for a coarse adjustment of the control parameters K_{P}, K_{I} and K_{D} and to use the at least one step response identified outside the calibration method following the training session in order to perform a fine adjustment of the control parameters K_{P}, K_{I} and K_{D}.

It is moreover conceivable for the computing unit to be configured to generate a second step response. For this purpose, the computing unit can be configured so that after the physiological data PD(t) have stabilized following the abrupt change in the manipulated variable, it switches the assistance over from u₂ to u₁ and waits again until the exertion data BD(t) or the physiological data PD(t) have stabilized. The control parameters K_{P}, K_{I} and K_{D} can differ when the assistance u(t) increases or decreases.

The computing unit 3 can be configured to adjust, on the basis of the optimization algorithm 11 (see FIG. 2), the coefficients aₓᵢ, the summand a₁₀, the delays τₓᵢ and the delay T after the training session, making use of the exertion data BD(t) ascertained in a plurality of training sessions and the physiological data PD(t) ascertained in the plurality of training sessions as well, optionally, as the altitude h(t) ascertained in the plurality of training sessions, the temperature Temp(t) ascertained in the plurality of training sessions and/or the incline N(t) ascertained in the plurality of training sessions in order to take an underlying fitness of the person 8 into consideration. For this purpose, the computing unit 3 can be configured to adjust the coefficients aₓᵢ, the summand a₁₀, the delays τₓᵢ and the delay T after the training session with the optimization algorithm 11, which has the steps of: a) specifying in each case a plurality of discrete values for each of the coefficients aₓᵢ, for the summand a₁₀, for each of the delays τₓᵢ, and for the delay T; b) setting aₓᵢ, a₁₀, τₓᵢ and T to one of the values; c) calculating mPD(t+T) on the basis of the model; d) calculating a modelling error between the measured physiological data PD(t+T) and mPD(t+T) for a plurality of t; e) repeating steps b) to d) for all combinations of the values; f) choosing those values for aₓᵢ, a₁₀, τₓᵢ and T that result in the lowest modelling error. Underestimation errors can be weighted 11 more strongly than overestimation errors in step d).

As can be seen from FIG. 2, the computing unit 3 can be configured to adjust, with an algorithm for adjusting a current fitness 12, the coefficients aₓᵢ and the summand a₁₀ during a training session, making use of the exertion data BD(t) ascertained in the training session and the physiological data PD(t) ascertained in the training session as well, optionally, as the altitude h(t) ascertained in the training session, the temperature Temp(t) ascertained in the training session and/or the incline N(t) ascertained in the training session in order to take the current fitness of the person 8 into consideration. For this purpose the computing unit can, for example, be configured to determine, with the algorithm for adjusting the current fitness 12, a difference Diff(t)=mPD(t)−PD(t) between the prediction of the physiological data mPD(t) and the measured physiological data PD(t), and if the difference Diff(t) exceeds a threshold value Threshold₁>0, to correct the coefficients aₓᵢ by adding a respective constant const₁ₓᵢ, as well as to correct the summand a₁₀ by adding a constant const₁₀ and, if the difference Diff(t) falls below a threshold value Threshold_{M}<0 to correct the coefficients aₓᵢ by adding a respective constant cons_{Mxi} as well as to correct the summand a₁₀ by adding a constant const_{M0}.

The coefficients aₓᵢ ascertained in the optimization algorithm 11 as well as delays τₓᵢ and T and the coefficients aₓᵢ ascertained in the algorithm for adjusting the current form 12 as well as the summand a₁₀ ascertained in the optimization algorithm 11 and in the algorithm for ascertaining the current fitness are used to prepare the prediction mPD(t+T) in a step 10. The prediction mPD(t+T) is the control variable in the control unit 4 and the manipulated variable is the assistance u(t).

The exertion data BD(t) can, for example, be a power, in particular a pedalling power in the case of a bicycle, in particular of an electric bicycle, or in the case of a bicycle ergometer, a running power, a rowing power, a speed, a torque, a rotation speed, an angular speed and/or a knee abduction torque. If the training device 2 is the bicycle or the bicycle ergometer, the power 9 that is applied by the person 8 during the training and absorbed by the training device 2 is a pedalling power. The training device 2 can, for example, also be a rowing ergometer or a rowing boat, and the exertion data could be the rowing power. The training device could also be an abductor/adductor machine, and the exertion data could be a knee abduction torque.

The assistance unit 6 can, for example, include an electric motor, a gearbox and/or a brake. The assistance u(t) applied by the assistance unit 6 can be positive, whereby the training is assisted, and/or negative, whereby the training is made more difficult. The electric motor is an example of the assistance unit 6 that is configured to assist the training. In this case, the assistance u(t) could, for example, be a power applied by the electric motor. It is alternatively conceivable that in the case in which the exertion data BD(t) are the power, the control unit 4 is configured to determine the power P_{M} of the electric motor in accordance with P_{M}(t)=u(t)*K*BD(t). The factor K indicates what maximum motor assistance is possible. K can, for example, be from 1 to 5 and in particular is 3. A brake of, for example, a bicycle ergometer is an example of the assistance unit that is configured to make the training more difficult. In this case, the assistance could, for example, be a braking power. An example of an assistance unit that is configured to support the training and to make it more difficult is the electric motor that is configured to perform recovery, i.e. to convert a pedalling power of the person into electrical current. The assistance unit 6 can be configured to control the assistance u(t) in small increments. For example, increments of a maximum of 3%, in particular a maximum of 1.5% or a maximum of 1%, are conceivable. It is the case here that 100% corresponds to a maximum assistance u(t) in the case that the assistance unit is configured to assist the training. In the case where the assistance unit is configured to make the training more difficult, −100% corresponds to a maximum opposition to the training.

The physiological data PD(t) can include a heart rate, a heart rate variability, an electrocardiogram, an oxygen saturation of the blood, a blood pressure, a neurological activity, in particular an electroencephalography, an adduction, in particular a knee adduction, and/or a knee bend. The adduction and/or the knee bend can, for example, be determined with a plurality of inertial measuring units attached to the person 8, which are configured to determine acceleration values and/or rotation data.

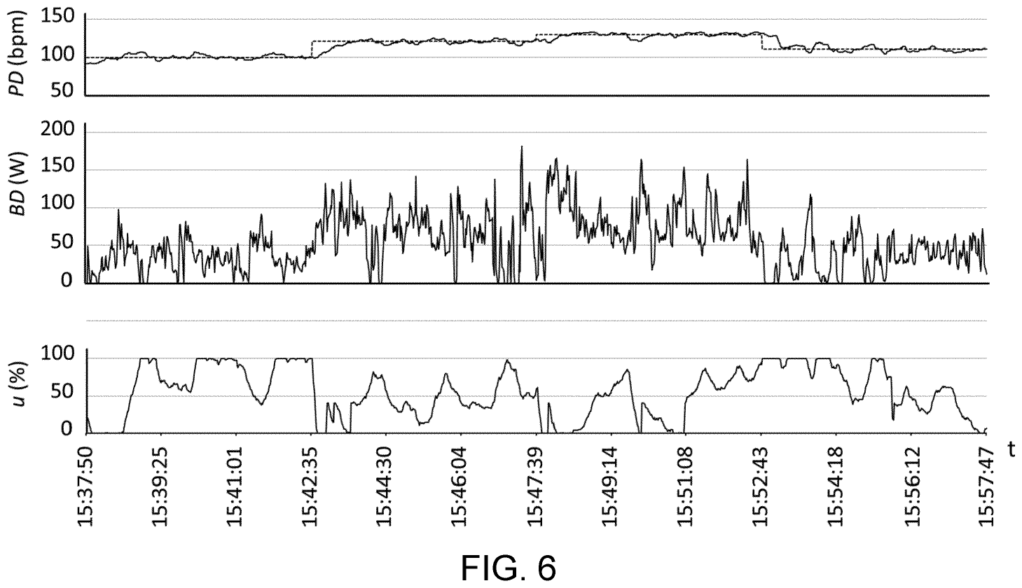

The physiological data PD(t), the exertion data BD(t) and the assistance u(t) of a training session carried out with an electric bicycle as the training device 2 are plotted in FIG. 6. The physiological data PD(t) are the heart rate in beats per minute (bpm). The heart rate can, for example, be measured with the body sensor 7 that is fitted in a chest strap. The exertion data BD(t) are the pedalling power in watts. The pedalling power can, for example, be determined by measuring the torque and the angular speed. To obtain a particularly high quality of the torque, the torque according to FIG. 6 was measured with a torque sensor supplied by Innotorq, as is, for example, described in WO 2015/028345 A1. The angular speed was measured through measurement of the rotation of a pole ring with a magnetic field sensor. The assistance unit 6 according to FIG. 6 is the electric motor of the electric bicycle, whose assistance is controlled from 0% to 100%. In the case in which the electric motor can perform recovery, the assistance can be controlled from −100% to 100%. The dashed line in the upper plot in FIG. 6 represents the reference variable. It can be seen that the reference variable can change over time. It can also be seen that the measured heart rate is at all times a good approximation of the reference variable.

### LIST OF REFERENCE NUMERALS

1 Apparatus
2 Training device
3 Computing unit
4 Control unit
5 Exertion measuring apparatus
6 Assistance unit
7 Body sensor
8 Person
9 Power
10 Preparation of the prediction mPD(t+T)
11 Optimization algorithm
12 Algorithm for adjusting the current fitness
13 Tangent at inflection point
BD(t) Exertion data
PD(t) Physiological data
mPD(t+T) Prediction of the physiological data
u Assistance
t Time
T_{U} Delay duration
T_{V} Settling duration
T₀ Time of the abrupt change in the assistance

What is claimed is:

1. An apparatus for controlling a training device, the apparatus comprising:
the training device configured to absorb a mechanical power applied by a person undertaking physical training, wherein the training device comprises an assistance unit configured to assist the training and/or to make the training more difficult, wherein the training device comprises an exertion measuring apparatus configured to measure mechanical exertion data BD(t) of an effort applied by the person during the training, wherein t is the time,
a body sensor configured to measure physiological data PD(t) of the body of the person,
a computing unit in which a mathematical model in the form mPD(t+T) is stored, wherein the computing unit (3) is configured, with an optimization algorithm, to adjust mPD(t+T) and the delay T individually for each person in such a way that mPD(t+T) approaches the measured physiological data PD(t+T), and to prepare a prediction mPD(t+T) of the physiological data PD(t+T) on the basis of the model, and
a control unit (4) that is configured to provide a predetermined reference variable for the physiological data PD(t), to take the prediction mPD(t+T) as a control variable, and to control an assistance u(t) of the assistance unit (6) as a manipulated variable.

2. The apparatus according to claim 1, wherein
$$m PD ( t + T ) = {a}_{10} + {\sum }_{x} {B}_{x} ( t ) \\ and \\ {B}_{1} ( t ) = {\sum }_{i = 1}^{j} {a}_{1 i} * ( {\sum }_{d = 0}^{{D}_{i}} BD ( t - {τ}_{1 i} - d * {K}_{i} ) / ( {D}_{i} + 1 ) ) \\ and \\ {B}_{2} ( t ) = {\sum }_{i = 1}^{k} {a}_{2 i} * PD ( t - {τ}_{2 i} )$$
apply,
wherein the computing unit is configured, with the optimization algorithm, to adjust the coefficients aₓᵢ, the summand a₁₀ and the delays τₓᵢ at least partially for each person individually in such a way that mPD(t+T) approaches the measured physiological data PD(t+T).

3. The apparatus according to claim 2, wherein the training device comprises an altimeter configured to measure the altitude h(t) of the training device, and
$${B}_{3} ( t ) = {\sum }_{i = 1}^{l} {a}_{3 i} * h ( t - {τ}_{3 i} )$$
in the model.

4. The apparatus according to claim 2, wherein the training device comprises a temperature sensor configured to measure the temperature Temp(t) in the surroundings of the training device (2), and
$${B}_{4} ( t ) = {\sum }_{i = 1}^{m} {a}_{4 i} * Temp ( t - {τ}_{4 i} )$$
in the model.

5. The apparatus according to claim 2, wherein the training device comprises an inclinometer configured to measure an incline N(t) of the training device, and
$${B}_{5} ( t ) = {\sum }_{i = 1}^{n} {a}_{5 i} * N ( t - {τ}_{5 i} )$$
in the model.

6. The apparatus according to claim 1, wherein the computing unit is configured to prepare the prediction mPD(t+T) for the time T that lies at least T=5 s in the future.

7. The apparatus according to claim 2, wherein the computing unit is configured to adjust, based on the optimization algorithm, the coefficients aₓᵢ, the summand a₁₀, the delays τₓᵢ and the delay T after the training session, making use of the exertion data BD(t) ascertained in a plurality of training sessions and the physiological data PD(t) ascertained in the plurality of training sessions, as well as, optionally, of the altitude h(t) ascertained in the plurality of training sessions, the temperature Temp(t) ascertained in the plurality of training sessions and/or the incline N(t) ascertained in the plurality of training sessions, in order to take an underlying fitness of the person (8) into consideration.

8. The apparatus according to claim 7, wherein the computing unit is configured to adjust the coefficients aₓᵢ, the summand a₁₀, the delays τₓᵢ and the delay T after the training session with the optimization algorithm which comprises the steps of:
a) specifying in each case a plurality of discrete values for each of the coefficients aₓᵢ, for the summand a₁₀, for each of the delays τₓᵢ, and for the delay T;
b) setting aₓᵢ, a₁₀, τₓᵢ and T to one of the values;
c) calculating mPD(t+T) based on the model;
d) calculating a modelling error between the measured physiological data PD(t+T) and mPD(t+T) for a plurality of t;
e) repeating steps b) to d) for all combinations of the values; and
f) choosing those values for aₓᵢ, a₁₀, τₓᵢ and T, that result in the lowest modelling error.

9. The apparatus according to claim 8, wherein underestimation errors are weighted more strongly than overestimation errors in step d).

10. The apparatus according to claim 2, wherein the computing unit is configured to adjust, with an algorithm for adjusting a current fitness, the coefficients aₓᵢ and the summand a₁₀ during a training session, making use of the exertion data BD(t) ascertained in the training session and the physiological data PD(t) ascertained in the training session, as well as, optionally, of the altitude h(t) ascertained in the training session, the temperature Temp(t) ascertained in the training session and/or the incline N(t) ascertained in the training session, in order to take the current fitness of the person into consideration.

11. The apparatus according to claim 10, wherein the computing unit is configured to determine, with the algorithm for adjusting the current fitness, a difference Diff(t)=mPD(t)−PD(t) between the prediction of the physiological data mPD(t) and the measured physiological data PD(t), and if the difference Diff(t) exceeds a threshold value Threshold1>0, to correct the coefficients aₓᵢ by adding a respective constant const1ₓᵢ, as well as to correct the summand a₁₀ by adding a constant const₁₀ and, if the difference Diff(t) falls below a threshold value of ThresholdM<0 to correct the coefficients aₓᵢ by adding a respective constant constMₓᵢ, as well as to correct the summand a₁₀ by adding a constant const_{M0}.

12. The apparatus according to claim 1, wherein the control unit is a PID controller.

13. The apparatus according to claim 12, wherein the PID controller is configured to determine the assistance u(t) according to
$$u ( t ) = {K}_{P} * {f}_{1} ( e ( t ) ) + {K}_{I} * {\int }_{τ = 0}^{τ = t} {f}_{2} ( e ( τ ) ) d τ + {K}_{D} * \frac{d {f}_{3} ( e ( t ) )}{dt}$$
wherein KP, KI, and KD are control parameters, wherein e(t) is the control deviation at time t, wherein the functions f1(e), f2(e) and f3(e) are selected such that underestimation errors are weighted more strongly than overestimation errors.

14. The apparatus according to claim 13, wherein the computing unit is configured to carry out a calibration method in which a step response of the physiological data PD(t) or of the exertion data BD(T) is generated by an abrupt change in the manipulated variable, and
wherein the computing unit is configured to determine the control parameters KP, KI, and KD from the step response.

15. The apparatus according to claim 13, wherein the computing unit is configured to identify at least one abrupt change in the manipulated variable, and the resulting step response of the physiological data PD(t) or of the exertion data BD(T) after a training session, and
wherein the computing unit is configured to determine the control parameters KP, KI, and KD from the at least one step response.

16. The apparatus according to claim 1, wherein the exertion data BD(t) is a power, in particular a pedalling power in the case of a bicycle, in particular of an electric bicycle, or, in the case of a bicycle ergometer, a running power, a rowing power, a speed, a torque, a rotation speed, an angular speed and/or a knee abduction torque.

17. The apparatus according to claim 1, wherein the assistance unit comprises an electric motor, a gearbox, and/or a brake.

18. The apparatus according to claim 1, wherein the physiological data PD(t) comprise a heart rate, a heart rate variability, an electrocardiogram, an oxygen saturation of the blood, a blood pressure, a neurological activity, in particular an electroencephalography, an adduction, in particular a knee adduction, and/or a knee bend.
